(19) United States
(12) Patent Application Publication
(10) Pub. No.: US 2005/0148928 A1
(43) Pub. Date: Jul. 7, 2005

(54) Multiple layer tubing and method of making same

(75) Inventors: Adam H. Molina, Valencia, CA (US); Poonam S. Gulati, La Canada, CA (US); Bradley J. Enegren, Moorpark, CA (US); Eric P. Geismar, Encino, CA (US); Philip J. Hudak, Thousand Oaks, CA (US)

Correspondence Address: FOLEY & LARDNER, 2029 CENTURY PARK EAST, SUITE 3500, LOS ANGELES, CA, 90067, US

(73) Assignee: Medtronic MiniMed, Inc.

(21) Appl. No.: 10/749,468

(22) Filed: Dec. 31, 2003

(51) Int. Cl. A61M 31/00

(52) U.S. Cl. 604/93.01; 264/634; 428/36.91; 604/523

(57) ABSTRACT

A multiple layer tubing for conveying a fluidic media in a medical or laboratory environment includes at least one outer layer of a copolyester ether (COPE) material. The tubing also includes an inner layer of a material compatible with sensitive fluidic media and at least one intermediate layer for bonding the outer layer to the inner layer. The material of the inner layer may be a high density polyethylene or a polyurethane material. The material of the intermediate layer may be ethylene-vinyl acetate (EVA). With the use of a COPE outer layer, a multiple layer medical tubing may be made without polyvinylchloride (PVC) or PVC plasticizers. The multiple layer tubing may be made by extrusion. The three layers may be co-extruded, simultaneously, as part of an efficient manufacturing processes.

### BACKGROUND OF THE INVENTION

1. Field of the Invention

The present invention relates, generally, to multiple layer tubing, processes of making such tubing and systems in which such tubing is used. In particular embodiments, the multiple layer tubing has an outer layer made of an elastomeric copolyester ether (COPE).

2. Description of Related Art

In biological, laboratory or medical environments, tubing is used in a variety of contexts, such as to communicate fluidic media between medical devices or from a medical device to a patient. Various types of medications, nutrients or other treatment formulations may be dispensed to patients through medical tubing, depending upon the patient's needs. In some contexts, it may be necessary to communicate media through a tubing for a protracted period of time and/or the media communicated through the tubing may be highly sensitive to its environment.

For example, hospital or long-term care patients with certain diabetic conditions may be connected to an insulin supply through a medical tubing. Such patients may require dosages of sensitive insulin formulations over a protracted period of time. The tubing may connect, at one end, to a medical pump and reservoir, a gravity-operated reservoir (e.g., an intravenous IV bag), or other suitable regulating device. The other end of the tubing may connect to the patient, through a suitable intravenous connection, catheter, cannula, infusion set, or the like.

Insulin compatible tubing made of polyvinyl chloride (PVC) is described in U.S. Pat. No. 4,723,947 to Konopka (assigned to the assignee of the present application), which is incorporated herein by reference. In the Konopka patent, a tubing has an inner layer of polyethylene, for compatibility with insulin. However, Konopka employs an outer layer of flexible PVC. To provide the PVC material with sufficient flexibility to function as a medical tubing, the PVC material includes a plasticizer.

### SUMMARY OF THE DISCLOSURE

Embodiments of the present invention relate to a multiple layer tubing for conveying a fluidic media. In one example embodiment, the tubing may be employed in a medical or laboratory environment, to convey sensitive media, such as insulin formulations, to a patient. In other embodiments, the tubing may convey other suitable fluidic media between medical devices or from a medical device to a patient.

The multiple layer tubing includes at least one outer layer made of a copolyester ether (COPE) material. The tubing also includes an inner layer made of a material compatible with the fluidic media and at least one intermediate layer for bonding the outer layer to the inner layer. Thus, the multiple layer tubing according to an example embodiment is a three-layer structure having one inner layer, one intermediate layer and one outer COPE layer.

In one embodiment, the material of the inner layer comprises high density polyethylene. In another embodiment, the material of the inner layer comprises a polyurethane. The material of the intermediate layer may comprise ethylene-vinyl acetate (EVA).

With the use of a COPE outer layer, embodiments of the multiple layer tubing may be made without polyvinylchloride (PVC) or PVC plasticizers. Thus, certain embodiments of the tubing may be made PVC-free.

The multiple layer tubing may be made by extruding the layers. Each of the inner, intermediate and outer layers may be extruded. In one embodiment, the three layers are co-extruded, simultaneously, as part of an efficient manufacturing processes.

These and other features and advantages of embodiments of the invention will be apparent to those skilled in the art from the following detailed description of embodiments of the invention, when read with the drawings and appended claims.

### BRIEF DESCRIPTION OF THE DRAWINGS

Referring now to the drawings in which like reference numbers represent corresponding parts throughout:

FIG. 1 is a cross-section diagram of a multiple layer tubing according to an embodiment of the present invention.

FIG. 2 is a schematic diagram representing an example of a connection arrangement using a tubing according to an embodiment of the present invention.

### DETAILED DESCRIPTION OF PREFERRED EMBODIMENTS

In the following description of embodiments of the invention, reference is made to the accompanying drawings which form a part of the description, and in which is shown by way of illustration specific embodiments in which the invention may be practiced. It is to be understood that other embodiments may be utilized and structural changes may be made without departing from the scope of the present invention.

Tubing, according to embodiments of the present invention, may be employed in a variety of contexts, including hospital and laboratory environments. For example, embodiments of the present invention may be employed as catheter tubing, tubing for disposable injection sets, cannulas, infusion sets, or the like. Examples of catheter environments in which embodiments of the present invention may be employed are described in U.S. patent application Ser. No. 10/035,831 and in U.S. patent application Ser. No. 10/331,949, filed Dec. 30, 2002, each of which is incorporated herein by reference in its entirety. Further example environments of use of such tubing as delivery tubing for subcutaneous infusion sets are described in U.S. patent application Ser. No. 10/705,686, filed Nov. 10, 2003 (assigned to the assignee of the present application), which is incorporated herein by reference in its entirety.

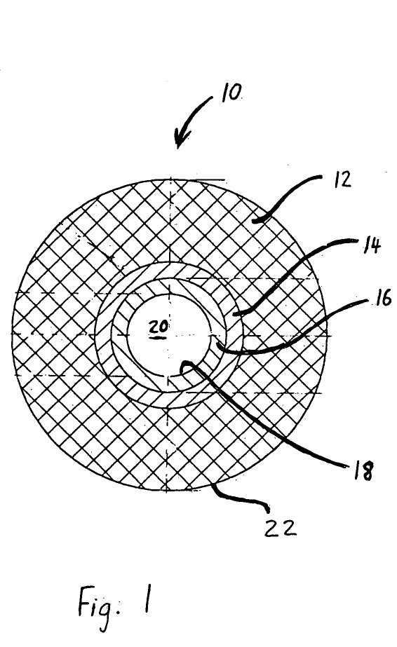

A tubing according to an example embodiment of the present invention comprises a layered structure, having multiple layers of materials, selected to provide certain beneficial qualities and characteristics as described below. A cross-section of a tubing 10 according to an embodiment of the present invention is shown in FIG. 1.

In FIG. 1, the tubing 10 has three layers, including an outer layer 12, an intermediate layer 14 and an inner layer 16. The inner layer 16 has an inner surface 18 that surrounds the hollow interior 20 of the tubing 10. The outer layer 12 has an outer surface 22, which, in some embodiments, defines the outer surface of the tubing 10.

The relative dimensions, including diameters and thicknesses, of the layers 12, 14 and 16 shown in FIG. 1, are examples and not limitations on the invention. Further embodiments may employ other dimensions suitable for the context of use. As a representative, non-limiting example of dimensions for a medical tubing shown in FIG. 1, the inner diameter of the inner layer 16 (i.e., the diameter of the inner surface 18) may be within the range of about 0.01 to about 0.025 inches and a thickness of about 0.005 to about 0.012 inches, preferably about 0.007 to about 0.009 inches. The intermediate layer 14 is relatively thin, on the order of about 0.0005 to about 0.005 inches and, preferably, about 0.001 to 0.002 inches. The thickness of the outer layer 16 is between about 0.005 and about 0.015 inches, preferably about 0.010 to about 0.012 inches. In one example embodiment, the completed tri-extruded tubing 10 will have an outside diameter of about 0.06 inches. Although other embodiments may employ other dimensions for the tubing layers and overall tubing, in some embodiments, the dimensions are selected to correspond to a type of infusion device. For example, the dimensions may be selected to allow the infusion pump to detect occlusions in the tubing, to deliver an accurately defined amount of media for each pump stroke, or to provide other features associated with an infusion device to which the tubing may connect.

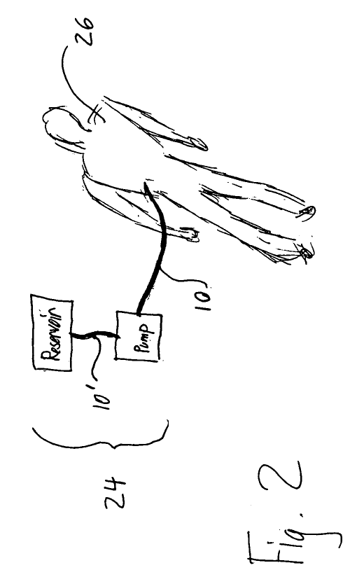

When in use, the ends of the tubing 10 are connected, through a suitable connector, for example, to a medical device or a patient. A simplified diagram of an example connection arrangement is shown in FIG. 2, wherein, a length of a medical tubing 10 connects an external pump and reservoir system 24 to a patient 26. The tubing 10 has one end 28 connected in fluid flow communication with the patient through a needle, Luer connector, an infusion set, an IV set, a catheter, a cannula or the like.

The tubing 10 has a second end 30 connected in fluid flow communication with the external system 24 through medical connectors, such as, but not limited to, Luer connectors or medical connectors as shown in U.S. Pat. No. 6,585,695, which is herein incorporated by reference in its entirety, or the like. Another example shown in FIG. 2 is represented by the further length of the tubing 10′ connected between two medical devices, such as between the reservoir and the pump of the system 24. In the further example, the two ends of the further length of tubing 10 may each have a connector suitable for connection to a medical device, including, but not limited to a Luer connector, a quick-release connector as shown in U.S. Pat. No. 5,545,152, medical connectors shown in U.S. Pat. No. 6,585,695 (both of which are herein incorporated by reference in their entireties), or the like.

With reference to FIG. 1, the outer layer 12 of the tubing 10 is selected and constructed to have certain beneficial qualities and characteristics relating to strength, durability, flexibility and connection capabilities. Tubing constructions have employed a polyvinylchloride (PVC) material with a plasticizer to provide some of these outer-layer characteristics. The PVC material is strong, durable and normally hard, but can be softened, to make flexible, by adding a plasticizer to the PVC formula. However, adding the plasticizer can allow the PVC and plasticizer to diffuse into the fluid. Many countries are seeking to ban the use of PVC in medical tubing. On the other hand, embodiments of the present invention employ a copolyester ether (COPE) material, as described in more detail below, and, thus, may avoid the use of PVC materials and the plasticizers used for softening PVC materials. In that regard, a medical tubing according to certain embodiments of the present invention may be made PVC-free.

The COPE outer layer 12 has suitable bonding characteristics for securing to connectors and other equipment. The outer layer 12 may be secured to a connector or other equipment by solvent bonding, adhesive bonding, clamping, flanging, ultrasonic welding, or the like. For solvent bonding, the COPE outer layer 12 is soluble in various suitable solvents, including, but not limited to cyclohexanone, ethylene dichloride, or the like. For adhesive bonding, the COPE outer layer 12 provides a suitable surface for applying an adhesive, such as an acrylic-based ultra-violet (UV) curable adhesive and a suitable barrier against diffusion of the adhesive through the tubing wall. For clamping, the COPE outer layer 12 is suitably flexible and abrasion-resistant to receive a clamping force from a typical tubing connector clamp.

In addition, the COPE outer layer 12 provides sufficient strength and flexibility characteristics to allow the tubing 10 to be used in a typical hospital or laboratory environment. The COPE outer layer 12 may be made in a sufficient thickness to provide a suitable resistance to tearing, cutting and puncture during ordinary use in a hospital or laboratory and, yet, allow the tubing 10 to be flexible and kink-resistant. Flexibility and kink-resistance can simplify storage and connection and can allow relative movement between the patient and the connected device.

The COPE outer layer 12 also provides a diffusion barrier which helps maintain the preservative concentrations (e.g., m-cresol phenol, or the like) in the media formulations. This property of the tubing is important as it helps maintain preservative levels in formulations, ensuring that the Antimicrobial Effectiveness requirements set in the United States Pharmacopeia and by the United States Food and Drug Administration are met.

Thus, the above advantages relating to strength, flexibility, capability of bonding to connectors or medical devices and diffusion barrier characteristics are provided by the COPE outer layer 12. Moreover, PVC-free embodiments may employ a tubing 10 having the COPE 12 outer layer in a PVC-free environment. While various manufacturing techniques may be employed to manufacture the tubing 10, according to various embodiments of the invention, COPE materials can be suitable for extrusion techniques. Thus, in extrusion embodiments described below, the COPE outer layer 12 may be formed by standard extrusion techniques.

An example of suitable COPE materials are the elastomer materials produced by Eastman Chemical Company under the product name ECDEL. The ECDEL elastomer identified as PCCE 9966 is employed in one example embodiment. However, other grades of ECDEL may be employed in other embodiments. While ECDEL had traditionally been employed for producing bags or films for wrapping products, embodiments of the present invention uniquely employs the elastomer material in a medical tubing and, in particular embodiments, in a multi-layer, PVC-free medical tubing.

The inner layer 16 of the multi-layer tubing 10 is made to have certain qualities and characteristics relating to compatibility, durability and flexibility. The material for the inner layer 16 must be compatible with the media to be conveyed through the tubing. The inner surface 18 of the inner layer 16 contacts the media during use. The material for the inner layer 16 must be compatible with the media over the expected period of use and, thus, must have sufficient strength and durability characteristics to withstand contact with the media over that period. In addition, material for the inner layer 16 is flexible, to allow the tubing 10 to be flexible.

In an example embodiment, the inner layer 16 of the multi-layer tubing 10 may be made of a high density polyethylene material. A high density polyethylene material can provide the above durability and flexibility properties and, also, be compatible with a variety of sensitive media, including sensitive protein formulas and/or formulas containing a phenol cresol preservative. As described in U.S. patent application Ser. No. 10/035,831 (which has been incorporated herein by reference in its entirety), some protein formulas, such as certain insulin formulas, can be sensitive to leaching or diffusion of molecules from or to the contacting surface of the tubing material, causing changes to the effectiveness of the formulation. High density polyethylene is compatible with many types of insulin and other protein formulations with respect to diffusion and leaching properties.

In addition, high density polyethylene can be manufactured within relatively small manufacturing tolerances. A capability of maintaining small manufacturing tolerances in an economical manufacturing process can be beneficial for certain tubing embodiments of the present invention. Small manufacturing tolerances can allow accurate tubing dimensions and, thus, accurate regulation of media through the tubing.

For example, it may be desirable, when conveying certain media through medical tubing, to accurately regulate the flow of the media to provide accurate dosages of concentrated drugs or other media. Accurate flow regulation may require a tubing diameter (diameter of the inner wall 18 of the inner layer 16) that is within a pre-defined manufacturing tolerance. An inner layer 16 made of high density polyethylene material can support relatively small manufacturing tolerances. In one embodiment, the material supports manufacturing tolerances of ±1/1000 inch.

While various manufacturing techniques may be employed to manufacture the tubing 10, according to various embodiments of the invention, high density polyethylene is suitable for extrusion techniques. By forming the inner layer 16 with an extrusion process, the inner layer 16 may be made with manufacturing tolerances as noted above in a relatively efficient manner. Other embodiments may employ one or more alternatives to high density polyethylene for the inner layer 16 including, but not limited to, polyurethane, bromobutyl rubber, chlorobutyl rubber, polypropylene, or the like.

The intermediate layer 14 of the multi-layer tubing 10 helps to join the outer layer 12 to the inner layer 16. The intermediate layer 14 is flexible, to allow the tubing 10 to be flexible. The intermediate layer 14 may have a soft, tacky characteristic, to frictionally bond to the outer and inner layers 12 and 16, once the layers are placed or formed on one another. In further embodiments, the intermediate layer 14 may be eliminated, such that the outer layer and inner layer are adhered together, directly, for example, with the use of frictional or self adhesive properties of those layers, curing, heating or other suitable bonding techniques.

In an example embodiment, the intermediate layer 14 may be made of an ethylene-vinyl acetate (EVA) material. An EVA material may be made to have the above properties and can contribute to simplifying the manufacturing of the tubing 10. The durometer of EVA material may be easily selected and changed during manufacturing processes, for providing the desired hardness/softness of the material. For example, a durometer of about 78 shore A may be used. Also, EVA is suitable for extrusion or co-extrusion with other layers, as described below. In such embodiments, once the layers are assembled or co-extruded together, the EVA intermediate layer 14 provides sufficient adhesion to inhibit delamination of the inner and outer layers 12 and 16. Alternatives to EVA for the intermediate layer 16 include polyurethane, or the like.

The tubing 10 may be manufactured in a variety of different manners. In accordance with an example embodiment of invention described above, the tubing 10 may be made by extruding the layers 12, 14 and 16. The layers may be co-extruded at the same time, by conventional co-extrusion processes. Thus, a co-extrusion technique can simplify and expedite the manufacturing process and allow the tubing 10 to be made economically and efficiently. In other embodiments, the layers may be extruded separately and assembled or extruded one-into-the-other. In yet other embodiments, the layers may be formed by other manufacturing techniques, including, but not limited to molding, layering sheets and rolling, or the like.

Each of the materials described above for layers 12, 14 and 16 may be made transparent (actually partially transparent). By forming the layers and, thus, the tubing 10 partially transparent, a person may view fluid flow, bubbles or blockages in the tubing, through the tubing wall.

While multi-layer tubing embodiments described above include three layers, other embodiments may employ additional layers, including, but not limited to additional high density polyethylene, EVA or COPE layers, or the like. For example, further embodiments may employ one or more additional layers to enhance strength and durability characteristics.

While embodiments described above relate to multi-layer tubing structures and processes, further embodiments may employ a single layer structure composed of a single layer of a COPE material, as described above. Yet further embodiments may employ multiple layers of a COPE material, without any further material layers. Yet further embodiments may employ multiple COPE layers interleaved with intermediate layers 14, as described above, but where the inner layer 16 is also made of a COPE material. For such embodiments, the single or multiple COPE layers (and any interleaved intermediate layers) may be formed by extrusion (co-extrusion for the multiple layer embodiments) or by other suitable manufacturing processes as described above. In each of those embodiments, the inner surface of a COPE inner layer (or a sole COPE layer, in the single layer embodiment) comes in contact with fluidic media passing through the tubing. Accordingly, testing of such embodiments with insulin or other sensitive media over time may be desired to evaluate the compatibility and stability of the COPE material and media in contact with each other over time. However, such embodiments would be applicable for contexts of use in which sufficient compatibility and stability exists. In some contexts, the texture or finish of the inner surface of the inner layer of COPE material and/or the particular formula of the COPE inner layer may be selected to enhance compatibility and stability. In yet further examples of such embodiments, the inner surface of a COPE inner layer may be coated with a suitable material to enhance compatibility and stability.

Also, while embodiments described above relate to the use of tubing 10 in medical environments, such as ambulatory, home, hospitals or laboratories, for connecting medical devices to each other or to patients, other embodiments may employ a tubing 10 for other contexts. For example, the tubing 10 may be employed for fluid flow applications in wet environments, sports environments, or the like.

The foregoing description of embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. A multiple layer tubing for conveying a fluidic media, the tubing comprising:
an inner layer made of a material compatible with the fluidic media;
an outer layer made of a copolyester ether (COPE) material; and
an intermediate layer for bonding the outer layer to the inner layer.

2. A tubing as recited in claim 1, wherein the material of the inner layer comprises high density polyethylene.

3. A tubing as recited in claim 2, wherein the material of the intermediate layer comprises ethylene-vinyl acetate (EVA).

4. A tubing as recited in claim 1, wherein the material of the inner layer comprises a polyurethane.

5. A tubing as recited in claim 1, wherein the material of the intermediate layer comprises ethylene-vinyl acetate (EVA).

6. A multiple layer tubing for conveying a fluidic media, the tubing comprising:
an inner layer made of a material compatible with the fluidic media;
at least one second layer for bonding a third layer to the inner layer; and
at least one third layer made of a copolyester ether (COPE) material.

7. A tubing as recited in claim 6, wherein the material of the inner layer comprises high density polyethylene.

8. A tubing as recited in claim 6, wherein the material of the inner layer comprises a polyurethane.

9. A tubing as recited in claim 6, wherein the material of each intermediate layer comprises ethylene-vinyl acetate (EVA).

10. A tubing as recited in claim 1, wherein the inner layer, the outer layer and the intermediate layer are each free of PVC.

11. A tubing as recited in claim 1, wherein the outer layer provides a barrier against diffusion of carbon dioxide.

12. A medical system for connection in fluid flow communication with a patient, the medical system comprising:
at least one medical device; and
a multiple layer tubing having one end connected to the at least one medical device and the other end for connection to the patient, the multiple layer tubing comprising an inner layer made of a material compatible with the fluidic media, at least one second layer for bonding a third layer to the inner layer; and at least one third layer made of a copolyester ether (COPE) material.

13. A medical system as recited in claim 12, wherein the at least one medical device comprises a reservoir containing an insulin formulation connected in fluid flow communication with the multiple layer tubing.

14. A medical system as recited in claim 13, wherein the at least one medical device further comprises a pump for providing a regulated flow of the insulin formulation from the reservoir and through the multiple layer tubing.

15. A tubing as recited in claim 13, wherein the material of the inner layer comprises high density polyethylene.

16. A tubing as recited in claim 13, wherein the material of the inner layer comprises a polyurethane.

17. A tubing as recited in claim 12, wherein the material of each second layer comprises ethylene-vinyl acetate (EVA).

18. A tubing as recited in claim 12, wherein the at least one second layer comprises no more than one second layer and the at least one third layer comprises no more than one third layer.

19. A tubing as recited in claim 12, wherein the inner layer, each outer layer and each intermediate layer are free of PVC.

20. A tubing as recited in claim 12, wherein at least one outer layer provides a barrier against diffusion of carbon dioxide.

21. A process of making a multiple layer tubing comprising:
forming an inner layer made of a material compatible with the fluidic media;
forming an outer layer made of a copolyester ether (COPE) material; and
forming an intermediate layer that bonds the outer layer to the inner layer.

22. A process as recited in claim 21, wherein forming an inner layer comprises extruding a tube-shaped inner layer of a first material.

23. A process as recited in claim 22, wherein forming an outer layer comprises extruding a tube-shaped outer layer of a COPE material.

24. A process as recited in claim 21, wherein forming an outer layer comprises extruding a tube-shaped outer layer of a COPE material.

25. A process as recited in claim 21, wherein forming the inner, outer and intermediate layers comprises co-extruding the layers at the same time.

26. A process as recited in claim 21, wherein the material of the inner layer comprises high density polyethylene or polyurethane.

27. A process as recited in claim 21, wherein the material for the second layer comprises ethylene-vinyl acetate (EVA).

28. A process as recited in claim 21, wherein forming an inner layer comprises extruding high density polyethylene or polyurethane.

29. A process as recited in claim 21, wherein forming a second layer comprises extruding ethylene-vinyl acetate (EVA).

30. A process as recited in claim 21, wherein the inner layer, the outer layer and the intermediate layer are each formed free of PVC.

31. A process as recited in claim 21, wherein the outer layer is formed of a material that provides a barrier against diffusion of carbon dioxide.

32. A tubing for conveying a fluidic media, the tubing comprising at least one layer of COPE material selected to be suitably compatible with the media.

33. A tubing as recited in claim 32, wherein the tubing comprises no more than one layer of COPE material.

34. A tubing as recited in claim 32, wherein the tubing consists essentially of no more than one layer of COPE material.

35. A tubing as recited in claim 32, wherein the tubing comprises a plurality of layers of COPE material.

36. A tubing as recited in claim 35, wherein an intermediate layer is interposed between each layer of COPE material, each intermediate layer for bonding two adjacent layers of COPE material.

37. A tubing as recited in claim 32, wherein the at least one layer of COPE material comprises a plurality of co-extruded layers of COPE material.

38. A process of making a tubing for conveying a media comprising:
selecting a COPE material that is suitably compatible with the media;
forming a tubing having at least one layer of the selected COPE material.

39. A process as recited in claim 38, wherein forming a tubing having at least one layer of the selected COPE material comprises forming no more than one layer of the selected COPE material.

40. A process as recited in claim 38, wherein forming a tubing having at least one layer of the selected COPE material comprises forming a plurality of layers of selected COPE material.

41. A process as recited in claim 40, further comprising forming an intermediate layer between at least two of the plurality of layers of selected COPE material, each intermediate layer for bonding two adjacent layers of selected COPE material.

42. A process as recited in claim 38, wherein forming a tubing having at least one layer of the selected COPE material comprises co-extruding a plurality of layers of selected COPE material.
